SEQUENCE LISTING (2 sequences)

SEQ ID NO: 1 - DNA, 20 bp, synthetic construct
  source 1..20: mol_type = other DNA; note = Forward primer
agagtttgatcmtggctcag

SEQ ID NO: 2 - DNA, 22 bp, synthetic construct
  source 1..22: mol_type = other DNA; note = Reverse primer
tacggytaccttgttacgactt

(19) United States
(12) Patent Application Publication
(10) Pub. No.: US 2025/0312314 A1
(43) Pub. Date: Oct. 9, 2025

(54) METHOD AND COMPOSITION FOR IMPROVING OR MAINTAINING SKIN HEALTH OF MAMMALS

(71) Applicant: NANJING NUTRABUILDING BIO-TECH CO., LTD., Nanjing (CN)

(72) Inventors: Ronghua YI, Nanjing (CN); Mingru WANG, Nanjing (CN); Qilin LIAO, Nanjing (CN)

(21) Appl. No.: 19/234,339

(22) Filed: Jun. 11, 2025

Related U.S. Application Data
(63) Continuation of application No. PCT/CN2023/138002, filed on Dec. 12, 2023.

(30) Foreign Application Priority Data
Dec. 12, 2022 (CN) 202211606160.1

(51) Int. Cl. A61K 31/4172; A61K 8/49; A61P 31/04; A61P 31/10; A61Q 17/00

(52) U.S. Cl. CPC A61K 31/4172; A61K 8/4946; A61P 31/04; A61P 31/10; A61Q 17/005

(57) ABSTRACT

Provided are a method and composition for improving or maintaining skin health of mammals and related use thereof in preparing personal care products, cosmetics or drugs for improving or maintaining skin health of mammals. The method or composition can inhibit the growth of microorganisms that cause skin problems, and/or promote the growth of microorganisms beneficial to skin, and/or eliminate or mitigate skin problems.

### CROSS REFERENCE TO RELATED APPLICATION

This application is a continuation application of International Patent Application No. PCT/CN2023/138002, filed on Dec. 12, 2023, which claims the priority to Chinese patent application No. 202211606160.1, filed on Dec. 12, 2022. The contents of International Patent Application No. PCT/CN2023/138002 and Chinese patent application No. 202211606160.1 are incorporated herein by reference in their entireties.

### REFERENCE TO AN ELECTRONIC SEQUENCE LISTING

The contents of the electronic sequence listing (SequenceListing.xml; Size: 3,177 bytes; and Date of Creation: Jun. 11, 2025) is herein incorporated by reference.

### TECHNICAL FIELD

The present disclosure belongs to the technical field of cosmetics and personal care products, and specifically relates to a method and composition for improving or maintaining skin health of mammals and the related use thereof in preparing personal care products, cosmetics or drugs for improving or maintaining skin health of mammals.

### BACKGROUND

There are all kinds of microorganisms clustered in the skin, which depend on each other and restrict each other to form a stable and harmonious biological barrier, and jointly maintain the microecological balance of the skin, which has important physiological functions. Therefore, skin microorganisms are important members of the skin microecosystem and play an important role in maintaining skin health.

Generally speaking, bacteria and fungi are important components of skin microorganisms, which jointly maintain the operation of skin. Most of the skin bacteria (>90%) are mainly actinomycetes (52%), Firmicutes (24%), Proteobacteria (16%) and Bacteroides (6%), while the main genus of skin fungi is Malassezia.

However, skin microorganisms may increase, decrease or even disappear abnormally due to some uncontrollable factors, and then the microbial state of skin is unbalanced, which will lead to a series of skin problems, such as atopic dermatitis and acne. For example, excessive growth of Malassezia, Propionibacterium acnes, Staphylococcus epidermidis, Cutibacterium spp., Corynebacterium spp. can cause inflammation. On the other hand, there are a large number of Streptococcus species in young individuals or individuals with more elastic skin; the decrease of Streptococcus, such as Streptococcus pneumoniae, Streptococcus infantis and Streptococcus thermophilus will lead to skin problems such as lack of elasticity and gloss, fine lines, sagging and dryness. Therefore, inhibiting the growth of these microorganisms causing skin problems and/or promoting the growth of microorganisms beneficial to skin, such as Streptococcus, can significantly improve the skin condition and maintain skin health.

In short, skin microbial imbalance will lead to skin damage, followed by inflammation and skin dryness, lack of elasticity and gloss, fine lines, sagging, aging and other problems. Ergothioneine (EGT) is a safe natural compound with excellent anti-inflammatory and antioxidant properties. In addition, it has been reported that EGT has a good regulatory effect on intestinal microorganisms (Matsuda, Ozawa et al. 2020), but there is no relevant research on the interaction between EGT and skin microorganisms, especially inhibiting the excessive growth of microorganisms causing skin problems, thus eliminating or inhibiting skin inflammation; or promoting the growth of microorganisms beneficial to skin, thereby protecting skin health.

### SUMMARY

To achieve the above objects, in one aspect, the present disclosure provides a method for improving or maintaining skin health of a mammal includes administering a composition to the mammal, wherein the composition includes an effective amount of ergothioneine or a pharmaceutically acceptable salt, acid, ester, analog or derivative thereof. In some embodiments, the composition further includes a pharmaceutically acceptable carrier.

In some embodiments, the improving or maintaining skin health of a mammal includes inhibiting the growth of microorganisms causing skin problems, and/or promoting the growth of microorganisms beneficial to skin, and/or eliminating or alleviating skin problems.

In some embodiments, the microorganisms causing skin problems are selected from one or more of the following: Staphylococcus epidermidis, Corynebacterium spp., Cutibacterium spp., Propionibacterium acnes, Malassezia; the microorganisms beneficial to skin are Streptococcus; the skin problems are selected from one or more of the following: lack of elasticity of skin, dry skin, desquamation, dull skin, fine lines, sagging, inflammation, peeling, acne, atopic dermatitis, eczema, chloasma, erythema, papules and itching. In some embodiments, the microorganisms beneficial to skin can be Streptococcus, such as Streptococcus pneumoniae, Streptococcus infantis, Streptococcus thermophilus; or other similar microorganisms. In some embodiments, Inflammation may be folliculitis, seborrheic dermatitis, etc.

In some embodiments, the composition is used for the preparation of personal care products, cosmetics or drugs.

In some embodiments, the composition is prepared in a form of lotion, cream, ointment, essence, paste, gel, solution, dispersion, spray, suspension, emulsion, foam, patch, powder, film, elixir, tincture, or liniment.

In some embodiments, the ergothioneine or a pharmaceutically acceptable salt, acid, ester, analog or derivative thereof is formulated to be administered in an amount of 1-1500 mg, preferably 1-1000 mg, 2-500 mg, 3-100 mg, 4-50 mg or 5-30 mg per day.

In some embodiments, the composition is administered topically, transdermally or subcutaneously. In some embodiments, the composition is administered orally and externally. For oral administration, the composition of the present disclosure can be in any suitable form, including solution, tablet, gel capsule, capsule or alternative nutritious food or nutritional supplement. For external administration, the composition will be applied to the skin part to be treated, especially the face, neck or hand, once or several times a day. The forms of the composition include skin lotion, skin softener, skin toner, skin cream, sunscreen, essence, mask pack, mask sheet, soap, shampoo, cleaning foam, cleaning lotion, cleaning cream, ointment, gel, massage cream and etc.

In another aspect, the present disclosure provides a composition including ergothioneine or a pharmaceutically acceptable salt, acid, ester, analog or derivative thereof, wherein the composition is used for improving or maintaining skin health of a mammal. In some embodiments, the composition further includes a pharmaceutically acceptable carrier.

In some embodiments, the composition inhibits the growth of microorganisms causing skin problems, and/or promotes the growth of microorganisms beneficial to skin, and/or eliminates or alleviates skin problems.

In some embodiments, the microorganisms causing skin problems are selected from one or more of the following: Staphylococcus epidermidis, Corynebacterium spp., Cutibacterium spp., Propionibacterium acnes, Malassezia; the microorganisms beneficial to skin are Streptococcus; the skin problems are selected from one or more of the following: lack of elasticity of skin, dry skin, desquamation, dull skin, fine lines, sagging, inflammation, peeling, acne, atopic dermatitis, eczema, chloasma, erythema, papules and itching. In some embodiments, the microorganisms beneficial to skin can be Streptococcus, such as Streptococcus pneumoniae, Streptococcus infantis, Streptococcus thermophilus; or other similar microorganisms. In some embodiments, Inflammation may be folliculitis, seborrheic dermatitis, etc.

In some embodiments, the composition is used for the preparation of personal care products, cosmetics or drugs. In some embodiments, the composition is prepared in a form of lotion, cream, ointment, essence, paste, gel, solution, dispersion, spray, suspension, emulsion, foam, patch, powder, film, elixir, tincture, or liniment.

In some embodiments, the ergothioneine or a pharmaceutically acceptable salt, acid, ester, analog or derivative thereof is formulated to be administered in an amount of 1-1500 mg, preferably 1-1000 mg, 2-500 mg, 3-100 mg, 5-50 mg, or 5-30 mg per day. In some embodiments, the composition is administered by a route such as topical, transdermal or subcutaneous administration. In some embodiments, the composition is administered orally and externally. For oral administration, the composition of the present disclosure can be in any suitable form, including solution, tablet, gel capsule, capsule or alternative nutritious food or nutritional supplement. For external administration, the composition will be applied to the skin part to be treated, especially the face, neck or hand, once or several times a day. The forms of the composition include skin lotion, skin softener, skin toner, skin cream, sunscreen, essence, mask pack, mask sheet, soap, shampoo, cleaning foam, cleaning lotion, cleaning cream, ointment, gel, massage cream and etc.

In another aspect, the present disclosure provides use of a composition in preparing personal care products, cosmetics or drugs for improving or maintaining skin health of a mammal, wherein the composition includes an effective amount of ergothioneine or a pharmaceutically acceptable salt, acid, ester, analog or derivative thereof. In some embodiments, the composition further includes a pharmaceutically acceptable carrier.

In some embodiments, the composition inhibits the growth of microorganisms causing skin problems, and/or promotes the growth of microorganisms beneficial to skin, and/or eliminates or alleviates skin problems. In some embodiments, the microorganisms causing skin problems are selected from one or more of the following: Staphylococcus epidermidis, Corynebacterium spp., Cutibacterium spp., Propionibacterium acnes, Malassezia; the microorganisms beneficial to skin are Streptococcus; the skin problems are selected from one or more of the following: lack of elasticity of skin, dry skin, desquamation, dull skin, fine lines, sagging, inflammation, peeling, acne, atopic dermatitis, eczema, chloasma, erythema, papules and itching. In some embodiments, the microorganisms beneficial to skin can be Streptococcus, such as Streptococcus pneumoniae, Streptococcus infantis, Streptococcus thermophilus; or other similar microorganisms. In some embodiments, Inflammation may be folliculitis, seborrheic dermatitis, etc.

In some embodiments, the composition is administered to the mammal in an amount of 1-1500 mg per day, preferably 1-1000 mg, 2-500 mg, 3-100 mg, 4-50 mg or 5-30 mg per day of ergothioneine or a pharmaceutically acceptable salt, acid, ester, analog or derivative thereof. In some embodiments, the composition is administered by a route such as topical, transdermal or subcutaneous administration. In some embodiments, the composition is administered orally and externally. In some embodiments, the composition is prepared in a form of lotion, cream, ointment, essence, paste, gel, solution, dispersion, spray, suspension, emulsion, foam, patch, powder, film, elixir, tincture, or liniment.

This summary is provided to introduce the selection of concepts in a simplified form, which are further described in the detailed description below. Summary is not intended to identify key features or essential features of the claimed subject matter, nor is it intended to be used to limit the scope of the claimed subject matter.

### BRIEF DESCRIPTION OF THE DRAWINGS

FIG. 1 is a comparison chart of Staphylococcus epidermidis counts after treatment with 1 μM α-GO or EGT of various concentrations.

FIG. 2 is a comparison chart of Corynebacterium spp. counts after treatment with 1 μM α-GO or EGT of various concentrations.

FIG. 3 is a comparison chart of Cutibacterium spp. counts after treatment with 1 μM α-GO or EGT of various concentrations.

FIG. 4 is a comparison chart of Propionibacterium acnes counts after treatment with 1 μM α-GO or EGT of various concentrations.

FIG. 5 is a comparison chart of Malassezia counts after treatment with 1 μM α-GO or EGT of various concentrations.

FIG. 6 is a comparison chart of relative abundance (RA) of Streptococcus in skin of subjects after applying pure base cream, 5% EGT base cream and 10% EGT base cream.

FIG. 7 is a comparative diagram elasticity, moisture and desquamation degree of skin of subjects after applying pure base cream, 5% EGT base cream and 10% EGT base cream.

### DETAILED DESCRIPTION OF THE EMBODIMENTS

Reference will now be made in detail to the preferred embodiments of the present disclosure, examples of which are further illustrated. While the present disclosure will be described in conjunction with the preferred embodiments, it will be understood that they are not intended to limit the present disclosure to these embodiments. To the contrary, the present disclosure is intended to cover alternatives, modifications and equivalents, which may be included within the spirit and scope of the present disclosure as defined by the claims.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” or their conjugations, refer to a situation where said terms are used in their non-limiting sense to mean that items following the word are included, but items not specifically mentioned are not excluded. It also encompasses the more limiting verb ‘to consist essentially of’ and ‘to consist of’.

As used herein, the term “mammal” or “subject” may be used interchangeably to refer to any animal to which the presently disclosed methods and compositions may be applied or administered. The animal may have an illness or other disease, but the animal does not need to be sick to benefit from the presently disclosed methods and compositions. As such, any animal may apply the disclosed compositions, or be a recipient of the disclosed methods. Although the animal subject is preferably a human, the methods and compositions of the present disclosure have application in veterinary medicine as well, e.g., for the treatment of domesticated species such as canine, feline, murine, and various other pets; farm animal species such as bovine, equine, ovine, caprine, porcine, etc.; and wild animals, e.g., in the wild or in a zoological garden, such as non-human primates.

As used herein, the term “administration” refers to the process of delivering a disclosed composition or active ingredient to a subject. The composition of the present disclosure is preferably administered by topical, transdermal or subcutaneous administration, but it can also be administered by other conventional routes to exert the desired effect.

As used herein, the term “effective amount” refers to an amount that is required to achieve the effect as taught herein. An effective amount herein includes, but is not limited to the amount necessary to improve or maintain skin health of mammals; and/or the amount necessary to inhibit the growth of microorganisms causing skin problems, and/or promote the growth of microorganisms beneficial to skin, and/or eliminate or alleviate skin problems. In accordance with the present disclosure, a suitable single dose size is that which, when administered one or more times over a suitable period of time, achieves the above-described effects.

As used herein, the term “pharmaceutically acceptable” means pharmaceutically, physiologically, cosmetically, or cosmetologically acceptable, and refers to those compositions or combinations of agents, materials, or compositions, and/or their dosage forms, which are within the scope of sound medical judgment, suitable for use in contact with the tissues of human beings and animals, compatible with other components of the composition, without excessive toxicity, irritation, allergic response, or other problem or complication, commensurate with a reasonable benefit/risk ratio.

As used herein, the term “carrier” may include liquid or solid fillers, diluents, excipients, solvents, encapsulating materials, buffers, stabilizers, preservatives, oils, surfactants, gelling agents, antioxidants, chelating agents, viscosity enhancers, ultraviolet absorbers, pH adjusters, pigments, fragrances, and the like. The carrier used in the present disclosure is preferably one that is suitable for use in personal care products, cosmetics, beauty products, or medicines.

The method of the present disclosure includes administering at least 1 mg, typically 1-1500 mg, preferably 1-1000 mg, 2-500 mg, 3-100 mg, 4-50 mg or 5-30 mg of ergothioneine or a pharmaceutically acceptable salt, acid, ester, analog or derivative thereof every day, depending on the specific preparation and form. The amount to be administered can also vary according to factors such as sensitivity, age, sex and weight of the subject, specific reaction, etc. One or more doses can be administered once or more times a day or at a suitable frequency for any period of time. For example, an effective dose can be administered daily for one day, several days, many days or indefinitely.

The following examples are illustrative of selected embodiments of the present disclosure and are not meant to limit the scope of the present disclosure.

### Example 1

Primary adult human skin fibroblasts were embedded in fibrin matrix to produce dermal equivalent. The dermal equivalent was cultured to allow fibroblasts to reconstruct matrix. Primary neonatal human keratinocytes were applied on the surface of dermal equivalent and cultured in liquid for 48 hours. Culture was carried out on the air-liquid interface (ALI) until a layered epidermis was formed.

Staphylococcus epidermidis, Corynebacterium spp., Cutibacterium spp., Propionibacterium acnes and Malassezia were cultured in proper culture medium and incubation conditions (37±2° C., 5±1% (v/v) CO₂, ≥95% RH). Inoculation buffer (GS25) was used to prepare inoculum containing about 1.1×10⁶ cfu mL⁻¹ microbial flora of the above bacteria. 10 μL of the mixture was used for skin (layered epidermis) colonization, and each unit was colonized with about 10⁴ cfu·cm⁻² of each bacterium. After that, the layered epidermis was incubated at 37±2° C., 5±1% (v/v) CO₂, ≥95% RH for 3±1 hours.

α-Glucan Oligosaccharide (α-GO for short) is a famous prebiotic, which can protect skin health by maintaining the balance of microbial population and strengthening the microbial barrier of skin, so the experiment took it as a control. Control (α-GO, 1 μM) and EGT (0 μM, 0.1 μM, 1 μM, 10 μM, 100 μM, 1000 μM) were applied on the layered epidermis respectively, and then incubated for 24±2 hours at 37±2° C., 5±1% (v/v) CO₂ and ≥95% RH. Then, the above five kinds of microorganisms were counted respectively to evaluate the inhibitory effect of α-GO and EGT on microbial growth: 8 mm live tissue slices were taken from layered epidermis, and the number of live bacteria (cfu·cm⁻²) was evaluated by incubation on appropriate selective solid medium. The choice of solid medium is based on the characteristics of microorganisms themselves.

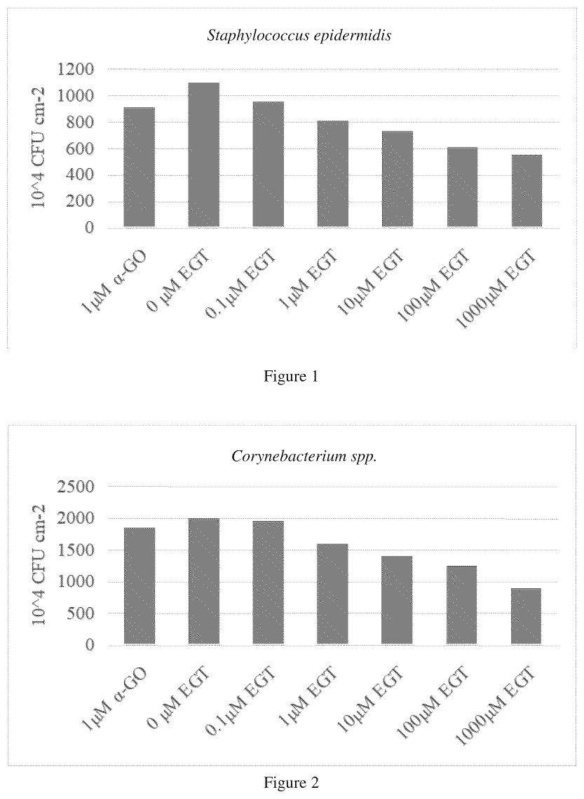

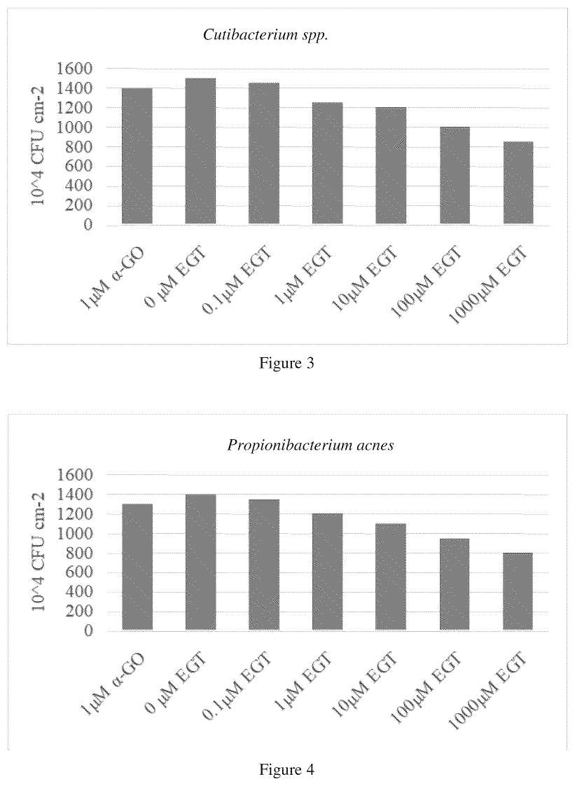

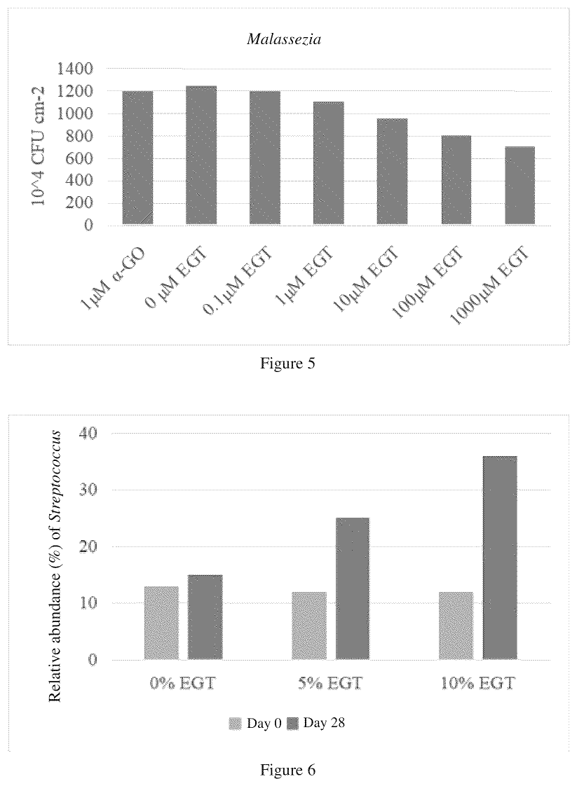

FIGS. 1-5 are comparison charts of Staphylococcus epidermidis, Corynebacterium spp., Cutibacterium spp., Propionibacterium acnes and Malassezia counts after treatment with 1 μM α-GO or EGT of various concentrations, respectively. As shown in FIGS. 1-5, the colonization of Staphylococcus epidermidis, Corynebacterium spp., Cutibacterium spp., Propionibacterium acnes and Malassezia was obviously inhibited in the layered epidermis after EGT treatment. The inhibition effect will be more significant with the increase of EGT concentration, so that the number of microorganisms can be maintained in a healthy state. In addition, at the same concentration (1 μM), EGT has a better inhibitory effect on the above five microorganisms than α-GO.

### Example 2

Thirty healthy women aged 20-59 were randomly divided into three groups. The first group (0% EGT, n=10) was applied with pure base cream as the control group, the second group (5% EGT, n=10) was applied with base cream mixed with 5% ergothioneine, and the third group (10% EGT, n=10) was applied with base cream mixed with 10% ergothioneine. The applying was performed once a day for 4 weeks. Skin microorganisms were collected on the 0 th and 28 th days respectively, and the elasticity, moisture and desquamation of the skin of the subjects were measured on the 28 th day. Sterile water was used to wash Participants' faces and then applied on solid trypsin soy agar (TSA) medium. Individual colonies were collected and cultured in liquid trypsin soy broth (TSB) medium at 37° C. for 72 hours.

Each sample was centrifuged and the precipitate was collected, and the microbial DNA in the precipitate was extracted with a specific kit. The purity and quantity of DNA were measured by spectrophotometer. The 16S rRNA gene of bacteria was amplified with the following primers: forward, 5′-AGAGTTTGATCMTGGCTCAG-3′ (SEQ ID NO: 1); reverse, 5′-TACGGYTACCTTGTTACGACTT-3′ (SEQ ID NO: 2). PCR was carried out in a 25 μL reactor, which contained 2 μL genomic DNA (10 ng/μL), 0.5 μL primers (10 μM), 12.5 μL 2×KAPA HiFi HotStart Ready Mix and 9.5 μL distilled water. The following PCR conditions were used: initially denaturing at 95° C. for 3 min; denaturing at 95° C. for 1 min, annealing at 55° C. for 1 min, and extending at 75° C. for 90 s for 30 cycles; finally, extending at 72° C. for 8 min. PCR products were sequenced on ABI-3730XL DNA sequencer, and 16S rRNA fragments were identified by NCBI Microbial Nucleotide BLAST and Mega-BLAST.

FIG. 6 is a comparison chart of relative abundance (RA) of Streptococcus in skin of subjects after applying pure base cream, 5% EGT base cream and 10% EGT base cream. As shown in FIG. 6, the Streptococcus in EGT base cream group is more abundant than that in pure base cream group, that is, EGT can promote the growth of microorganisms beneficial to skin, such as Streptococcus; and the effect of 10% EGT group is more obvious.

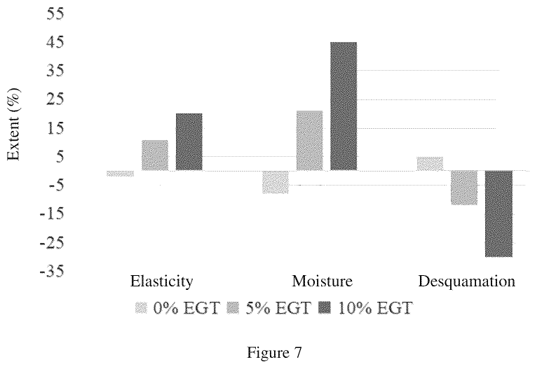

FIG. 7 is a comparative diagram elasticity, moisture and desquamation degree of skin of subjects after applying pure base cream, 5% EGT base cream and 10% EGT base cream. As shown in FIG. 7, EGT can significantly increase the skin elasticity and moisture of the subjects and avoid the problem of skin desquamation. The effect of 10% EGT group is more obvious. Compared with 0% EGT group, it increases skin elasticity by about 20% and skin moisture by about 45%, and reduces desquamation by about 30%.

Too much or too little Staphylococcus epidermidis will have adverse effects on the skin. Too much Staphylococcus epidermidis will lead to problems such as Cystic acne, topic dermatitis, folliculitis, sty, cellulitis, seborrheic dermatitis and eczema. Corynebacterium spp. will prevent the immune system from recognizing and clearing it and continue to reproduce, resulting in persistent infection, which will eventually cause skin redness, swelling, pain and even pustules. Propionibacterium acnes will decompose skin and sebum when it is over-propagated, resulting in skin infection and damage. At the same time, the bacteria can promote the oxidation of sebum, cause skin inflammatory reaction and oxidative stress, and thus lead to acne. The inventors found that EGT has a positive effect on the regulation of microbial flora, which can significantly inhibit excessive Staphylococcus epidermidis, and can also significantly inhibit the (excessive) growth of Corynebacterium spp., Cutibacterium spp., Propionibacterium acnes, Malassezia and other microorganisms that are prone to skin problems. It can promote the growth of Streptococcus, such as Streptococcus pneumoniae, Streptococcus infantis, Streptococcus thermophilus and other microorganisms beneficial to skin. It can also eliminate or alleviate skin problems, such as lack of elasticity of skin, dry skin, desquamation, dull skin, fine lines, sagging, inflammation, peeling, acne, atopic dermatitis, eczema, chloasma, erythema, papules, itching, folliculitis, seborrheic dermatitis, etc; for example, it can increase skin elasticity and moisture and avoid skin desquamation. In conclusion, EGT can repair skin injury, regulate skin microbial homeostasis, maintain and improve skin health, and improve skin condition and physiological function.

Although specific embodiments and examples of the present disclosure have been illustrated herein, it will be appreciated by those skilled in the art that any modifications and variations can be made without departing from the spirit of the present disclosure. The examples and illustrations above are not intended to limit the scope of the present disclosure. Any combination of embodiments of the present disclosure, along with any obvious their extension or analogs, are within the scope of the present disclosure. Further, it is intended that the present disclosure encompass any arrangement, which is calculated to achieve that same purpose, and all such variations and modifications fall within the scope of the appended claims.

What is claimed is:

1. A method for improving or maintaining skin health of a mammal, comprising administering a composition to the mammal, wherein the composition comprises an effective amount of ergothioneine or a pharmaceutically acceptable salt, acid, ester, analog or derivative thereof.

2. The method according to claim 1, wherein the improving or maintaining of the skin health of the mammal comprises at least one of:
inhibiting growth of microorganisms causing skin problems,
promoting growth of microorganisms beneficial to skin, or
eliminating or alleviating skin problems.

3. The method according to claim 2, wherein the microorganisms causing skin problems are selected from one or more of the following: Staphylococcus epidermidis, Corynebacterium spp., Cutibacterium spp., Propionibacterium acnes, or Malassezia; the microorganisms beneficial to skin are Streptococcus; the skin problems are selected from one or more of the following: lack of elasticity of skin, dry skin, desquamation, dull skin, fine lines, sagging, inflammation, peeling, acne, atopic dermatitis, eczema, chloasma, erythema, papules, or itching.

4. The method according to claim 1, wherein the composition is used for preparation of personal care products, cosmetics or drugs.

5. The method according to claim 1, wherein the composition is prepared in a form of lotion, cream, ointment, essence, paste, gel, solution, dispersion, spray, suspension, emulsion, foam, patch, powder, film, elixir, tincture, or liniment.

6. The method according to claim 1, wherein the ergothioneine or the pharmaceutically acceptable salt, acid, ester, analog or derivative thereof is formulated to be administered in an amount of 1 mg per day to 1500 mg per day.

7. The method according to claim 1, wherein the composition is administered topically, transdermally or subcutaneously.

8. The method according to claim 3, wherein the Streptococcus is Streptococcus pneumoniae, Streptococcus infantis, or Streptococcus thermophilus.

9. The method according to claim 3, wherein the inflammation is folliculitis or seborrheic dermatitis.

10. The method according to claim 1, wherein the composition further comprises a pharmaceutically acceptable carrier.

11. The method according to claim 1, wherein the composition is administered orally and externally.

12. The method according to claim 1, wherein the composition is applied to a portion of skin once or at least twice a day.
